(19) United States
(12) Patent Application Publication
(10) Pub. No.: US 2024/0181431 A1
(43) Pub. Date: Jun. 6, 2024

(54) MULTI-LAYER COMPOSITES AND ABSORBENT ARTICLES INCLUDING THE SAME

(71) Applicant: FIRST QUALITY RETAIL SERVICES, LLC, Great Neck, NY (US)

(72) Inventor: Susan O'Connell, Chesterbrook, PA (US)

(21) Appl. No.: 18/527,759

(22) Filed: Dec. 4, 2023

Related U.S. Application Data
(60) Provisional application No. 63/385,832, filed on Dec. 2, 2022.

(51) Int. Cl. B01J 20/28; A61F 13/535; A61F 13/538; B01J 20/26

(52) U.S. Cl. CPC B01J 20/28035; A61F 13/535; A61F 13/538; B01J 20/26; B01J 20/28028; A61F 2013/15414

(57) ABSTRACT

A nonwoven composite including at least two layers with each layer being made of nonwoven and/or tissue and having a lower side and an upper side. The lower side of a first layer of the at least two layers is attached to the upper side of an adjacent second layer of the at least two layers with a pattern of continuous attachment lines extending in a machine direction across a cross direction width of the nonwoven or composite.

### FIELD OF THE INVENTION

The present invention generally relates to nonwovens or composites, and in particular to multi-layer absorbent nonwovens or composites useful in hygienic absorbent articles.

### BACKGROUND

Diapers, training pants, briefs, sanitary pads and pantiliners have been made and sold for many years. In general, these products prevent accidental contact with body fluids or feces. The products typically include an absorbent core to retain fluids. The cores may be used with acquisition distribution layers (“ADL”) adjacent to, above or below the absorbent core. While generally effective in absorbing liquids, these cores may be thick, stiff, or uncomfortable. Absorbent cores frequently contain superabsorbent particles (“SAP”) to provide enhanced absorbency. The particles are small and sometimes difficult to immobilize without having a negative effect on absorbency of the particles. As SAP particles absorb moisture, they swell. The absorbency of SAP particles can become somewhat limited by lack of space for particles to swell.

While current nonwoven and composite technologies are useful, there is a continuing need to provide improved nonwovens or composites that are useful in absorbent products, including absorbent cores with increased pliability and ability to absorb moisture.

### SUMMARY OF THE INVENTION

A nonwoven or composite according to an exemplary embodiment of the present invention comprises: at least two layers with each layer being made of nonwoven and/or tissue and having a lower side and an upper side, wherein the lower side of a first layer of the at least two layers is attached to the upper side of an adjacent second layer of the at least two layers with a pattern of continuous attachment lines extending in a machine direction across a cross direction width of the nonwoven or composite.

An absorbent product according to an exemplary embodiment of the present invention comprises: a nonwoven or composite comprising: at least two layers with each layer being made of nonwoven and/or tissue and having a lower side and an upper side, wherein the lower side of a first layer of the at least two layers is attached to the upper side of an adjacent second layer of the at least two layers with a pattern of continuous attachment lines extending in a machine direction across a cross direction width of the nonwoven or composite; a backsheet; and a topsheet.

In an exemplary embodiment the attachment lines are from 1 to 3 mm wide and are spaced from 10 to 30 mm apart.

In an exemplary embodiment the nonwoven or composite comprises from 10 to 80% superabsorbent polymer particles by weight of the nonwoven or composite.

In an exemplary embodiment a basis weight of the nonwoven or composite ranges from 15 g/m² to 400 g/m².

In an exemplary embodiment a dry thickness of the nonwoven or composite ranges from 1 mm to 5 mm and the thickness of the nonwoven or composite when wet ranges from 10 mm to 125 mm.

### BRIEF DESCRIPTION OF THE DRAWINGS

FIG. 1 is a cross-section view of an absorbent nonwoven or composite according to an exemplary embodiment of the present invention;

FIG. 2 is a cross-section view of an absorbent product according to another exemplary embodiment of the present invention; and

FIG. 3 is a cross-section view of an absorbent nonwoven or composite according to an exemplary embodiment of the present invention when wet.

### DETAILED DESCRIPTION OF PREFERRED EMBODIMENTS

Nonwovens or composites according to exemplary embodiments of the present invention include at least two layers. A composite in accordance with exemplary embodiments of the invention may include, for example, at least one tissue layer and at least one nonwoven layer, or at least one layer of both nonwoven and tissue. A nonwoven in accordance with exemplary embodiments of the present invention may include, for example, at least one layer of a first nonwoven and a second layer of a second nonwoven, or at least two layers of the same nonwoven. Nonwovens are typically made from nylon, polyester, polyethylene, or polypropylene, and are made by mechanically, chemically, or thermally interlocking polymer fibers. Nonwovens used in diapers are typically made with the spun bonding process, which has good tensile strength and abrasive resistance. Thermal bonded nonwovens are also frequently utilized, as they are softer but they have lower resistance and strength. Through Air Bonded nonwovens which are more lofty, can also be used.

Tissue is known in the art and typically is made from natural fibers such as wood pulp. Suitable natural fibers include, but are not limited to softwood fibers, hardwood fibers, elephant grass, nettle, buntal, buri, soybean protein, milvet milk, abaca, bagasse, bamboo, coir, cotton, flax, linen, hemp, jute, kapok, kenaf, pina, raffia, ramie, sisal, oxidized natural fibers and combinations thereof.

The basis weight of the absorbent nonwoven, tissue, or composite may range from 15 g/m² to 400 g/m². The thickness of the absorbent nonwoven or composite when dry may range from 1 mm to 5 mm. The thickness of the absorbent nonwoven or composite when wet may range from 10 mm to 125 mm. The nonwovens, tissue, or composites may include conventional SAP technology. The amount of SAP may range from 5% to 85% or 10% to 80% or 20% to 70% or 39% to 60% by weight, based on the total weight of the absorbent product. There are commercially available SAPs with different properties, for example increased absorbency or faster absorbency relative to other SAPs. The present invention includes nonwovens, tissue, or composites with more than one type of SAP combined or applied in different regions of the absorbent product.

In an embodiment, the nonwoven or composite utilized in the present invention may be a fiber nonwoven or composite made from continuous fibers. As used herein, continuous means that the fiber is extruded from molten or dissolved polymer and continuously wound or spun in a pattern to make a nonwoven or composite material. The fibers or filaments may be made from polyolefins, such as polyethylene, polypropylene, polybutene, polylactic acid, polycaprolactone or polyesters and the like. The polymer is melted or dissolved in a solvent to make a liquid and passed through an orifice such as a spinneret. Upon exiting the orifice, solid particle material is combined into the liquid polymer and embeds in the polymer as it solidifies through cooling or evaporation of the solvent. The solid polymer particles may be SAP, odor control particles, skin health additives or combinations thereof. The solid particles may be fed from a hopper into the flowing liquid polymer exiting the spinnerets. Alternatively, the solid particles may be applied to the liquid polymer in a fluidized bed. The particles are immobilized through embedment into the polymer, entrapment within a fiber network or both. The amount of solid SAP particles is typically 50% or more, or 60% or more or 70% or more by weight of the total nonwoven or composite. The amount of odor control solid particles may range from 0.1% to 5% by weight of the nonwoven or composite. The amount of skin health additive may range from 0.01% to 5% by weight of the nonwoven or composite. The basis weight of the absorbent nonwoven or composite may range from 50 g/m² to 400 g/m². The thickness of the absorbent nonwoven or composite when dry may range from 1 mm to 5 mm. The thickness of the absorbent nonwoven or composite when wet may range from 10 mm to 125 mm.

By embedding the solid particles in the fibers or filaments, the particles are trapped or immobilized, thereby preventing leaking out of the product without deleteriously effecting the function of the particles and reducing the amount of adhesive utilized in the product. Alternatively, the solid particles may be trapped in a network of fibers formed from spinning or forming the nonwoven or composite. The network is formed from continuous fibers crossing over each other in the forming process.

The absorbent nonwoven or composite may include continuous elastic or stretchable fibers in the cross direction and/or the machine direction. Suitable elastic fibers include LYCRA® from DuPont, GLOSPAN® from Globe, VISTAMAXX® from ExxonMobil and the like. The amount of elastic fiber may range from 5% to 60% or 10% to 50% or 20% to 40% based on the total weight of the nonwoven or composite.

Layers of the nonwoven or composite may be stacked on top of each other and attached through ultrasonic bonding, adhesive, needle punching and the like. The layers may be attached with attachment lines that extend in the machine direction (Y-direction as indicated in the figures) that are 1-3 mm wide as measured in the cross-direction (X-direction as indicated in the figures), spaced 10-30 mm apart along the cross-direction, attaching the lower side of one layer to the upper side of an adjacent layer. Combining multiple layers of the nonwoven, tissue, or composite may result in increased basis weight, absorbency, cushioning and the like. When additional layers of nonwoven or composite are utilized, for example three or more layers, the layers are attached to adjacent layers with a pattern of attachment lines that is staggered or offset to the approximate center of the pattern on attachment lines on the adjacent layer. This creates a honeycomb appearance as viewed along the z-direction. When pressed from the sides, the nonwoven, tissue, or composite layers will separate in between the attachment lines and form a gap reminiscent of a honeycomb. This space enables SAP to swell without limiting absorbent capacity. The nonwoven or composite may be formed into rolls which are cut to form absorbent cores or materials utilized in absorbent products. As used herein, the term “absorbent core” refers to a material or combination of materials suitable for absorbing, distributing, and storing aqueous fluids such as urine, blood, menses, and water found in body exudates. The size and shape of the absorbent core can be altered to meet absorbent capacity requirements, and to provide comfort to the wearer/user. The length of the absorbent core may range from about 10 cm to about 60 cm. The width of the absorbent core may range from about 4 cm to about 20 cm.

When a second absorbent core is utilized, the second absorbent core may be the same size as the first core, smaller than the first core, or larger than the first core. The absorbent core suitable for use in the present invention can be any liquid absorbent material known in the art for use in absorbent articles, provided that the liquid absorbent material can be configured or constructed to meet absorbent capacity requirements. The absorbent nonwoven or composite may be useful in sanitary napkins, pantiliners, incontinence pads, diapers, briefs, bladder control pads and the like.

In exemplary embodiments, the nonwoven or composite may be combined with an acquisition distribution layer (ADL) for distributing liquids to the fluid-absorbent core. The ADL may be made entirely of conventional fibrous materials with little absorbency, but in some embodiments includes water-absorbent polymer particles or other absorbent materials. The fibrous material may be hydrophilic, hydrophobic or can be a combination of both hydrophilic and hydrophobic fibers. The fibrous material may be derived from natural fibers, synthetic fibers or a combination of both. Suitable ADLs are formed from cellulosic fibers and/or modified cellulosic fibers and/or synthetics or combinations thereof. Thus, suitable ADLs may contain cellulosic fibers, in particular wood pulp fluff. Modified cellulosic fibers may be utilized for fluid acquisition and distribution. Examples of modified cellulosic fibers are chemically treated cellulosic fibers, especially chemically stiffened cellulosic fibers. The basis weight of cellulosic fibers and modified cellulosic fibers may range from about 50 to about 200 g/m².

Suitable acquisition-distribution layers may further include synthetic fibers. Hydrophilic synthetic fibers may be obtained by chemical modification of hydrophobic fibers, such as by surfactant treatment of hydrophobic fibers. The surface of the hydrophobic fiber can be rendered hydrophilic by treatment with a nonionic or ionic surfactant, e.g., by spraying the fiber with a surfactant or by dipping the fiber into a surfactant.

Alternatively, a bundle of synthetic fibers acting as an ADL loosely distributed on top of or below the fluid-absorbent core may be used. Suitable synthetic fibers include, for example, copolyester, polyamide, copolyamide, polylactic acid, polypropylene or polyethylene, viscose or blends thereof. Bicomponent fibers may also be used. In exemplary embodiments, the synthetic fiber component may be composed of either a single fiber type with a circular cross-section or a blend of two fiber types with different cross-sectional shapes.

The ADL basis weight may range from about 20 g/m² to about 200 g/m², depending on the concentration of water-absorbent polymer particles. The length of the ADL may range from about 6 cm to about 60 cm. The width of the ADL may range from 4 cm to 15 cm.

In exemplary embodiments, a core wrap may assist with containment and integrity of the absorbent core components. The core wrap may be bonded to the absorbent core. Bonding of the core wrap to the absorbent core may occur via any means known to one of ordinary skill, such as, but not limited to, adhesives. The core wrap may be composed of separate sheets of material (such as an upper core wrap and a lower core wrap) which can be utilized to partially or fully encompass the absorbent core and which can be sealed together using a sealing means such as an ultrasonic bonder or other thermochemical bonding means, or the use of an adhesive. The core wrap may include, but is not limited to, natural and synthetic fibers such as polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers may include wool, cotton, flax, hemp, and wood pulp. The material forming the core wrap may be selected from meltblown-spunbond-meltblown fabric, spunbond fabric, meltblown fabric, coform fabric, carded web, bonded-carded web, bicomponent spunbond fabric, spunlace, tissue, and combinations thereof. Further, the core wrap may be made of a spunbond-meltblown-spunbond (“SMS”) material, such as a 9 g/m² spunbond-meltblown-spunbond material.

The core wrap may be less hydrophilic than the absorbent core, but sufficiently porous to permit body fluids to penetrate through the core wrap to reach the absorbent core. The core wrap may have sufficient structural integrity to withstand its own wetting and the wetting of the absorbent core. In order to support this functional property of the core wrap, a wet strength agent may be applied to the core wrap. A non-limiting example of a wet strength agent may be Kymene 6500 (557LK) or equivalent available from Ashland Inc. of Ashland, Ky., U.S.A. Similarly, a surfactant may be included in the core wrap to promote hydrophilicity.

The upper and lower core wraps may be attached at the sides adjacent to the core (sandwich attachment) or in a C wrap configuration. With the C wrap configuration, the upper core wrap may fold around and under the bottom of the core, where it is attached to the lower core wrap. Alternatively, the lower core wrap may fold around and over the upper of the core, where it is attached to the upper core wrap. In some embodiments, the absorbent system does not include a core wrap.

In use, the nonwovens or composites of exemplary embodiments of the present invention are placed on a top surface of a backsheet. Backsheets are materials that generally are liquid impermeable but may be moisture vapor permeable (breathable). Backsheets are used in absorbent products on a surface of the product that is distal to the user's body. The backsheet can be made of any known or otherwise effective backsheet material, provided that the backsheet prevents external leakage of exudates absorbed and contained in the protective underwear. Flexible materials suitable for use as the backsheet include, but are not limited to, woven and nonwoven materials, laminated tissue, polymeric films such as thermoplastic films of polyethylene and/or polypropylene, microporous films, composite materials such as a film-coated nonwoven material, or combinations thereof, as is well known in the art of making absorbent products, such as sanitary napkins, pantiliners, incontinence pads, and the like.

The absorbent system is typically attached to the backsheet with an adhesive. Suitable adhesives are known in the art and include hot melt adhesives, emulsion polymer adhesives and the like.

A topsheet or cover is placed on top of the absorbent system and attached to the core or insert and backsheet with adhesive, ultrasonic bonding or combinations thereof, forming a chassis. Suitable topsheets are compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. A suitable topsheet can be made of various materials, such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making absorbent products such as sanitary napkins, pantiliners, incontinence pads, protective underwear and the like.

Elastic side panels may be attached to the chassis to form diapers or adult protective underwear. Any elastic side panel known in the art of absorbent articles may be useful. Suitable elastic side panels include laminates of elastic films with nonwovens, laminates of elastic strands with nonwovens and the like. The elastic panels may be attached to the chassis by adhesive, ultrasonic bonding or a combination thereof. The length, width and shape of the side panels may be designed to make products of different sizes. Products with side panels may have a more underwear like appearance.

The side panels are then attached to the insert/core. A portion of each side panel is left unattached to form leg openings. The side panels may be attached to the insert/core with glue, adhesive, ultrasonic bonding or combinations thereof. The side panels may be attached to the insert/core at various angles to create a more garment like fit.

As is known in the art, hooks and loops may be used on articles in accordance with exemplary embodiments of the present invention. Nonwoven materials may function as the loops.

In exemplary embodiments, the hook fasteners may be made up of separate hook elements or may be integral with the side panels. In this regard, the hook elements may be bonded to the side panels by adhesive, ultrasonic, thermal bonding or the like. Alternatively, the hook elements may be intimately joined with the material that forms the side panels. Such intimate bonding of hook elements with a layer of material may be accomplished by feeding extruded plastic and a sheet of material through a nip formed by a first roller having pins and a second roller having corresponding cavities. As the molten plastic is forced through the nip, it flows into the cavities of the second roller and also into pores in the sheet of material. The plastic in the cavities cools and hardens so as to form a hook sheet. At the same time, the sheet of material is intimately joined to and becomes an integral part of the hook sheet so as to form a laminated structure. This process is disclosed in U.S. Pat. No. 5,518,795, the contents of which are incorporated herein by reference in their entirety. The hook elements may be arranged on the side panels in longitudinally extending strips that are laterally spaced from one another. Alternatively, the hook elements may be arranged in a pattern of geometric shapes or lines. Desirably, the hook elements are arranged on an inelastic material in order to improve ease of processing and the shear strength of the seam.

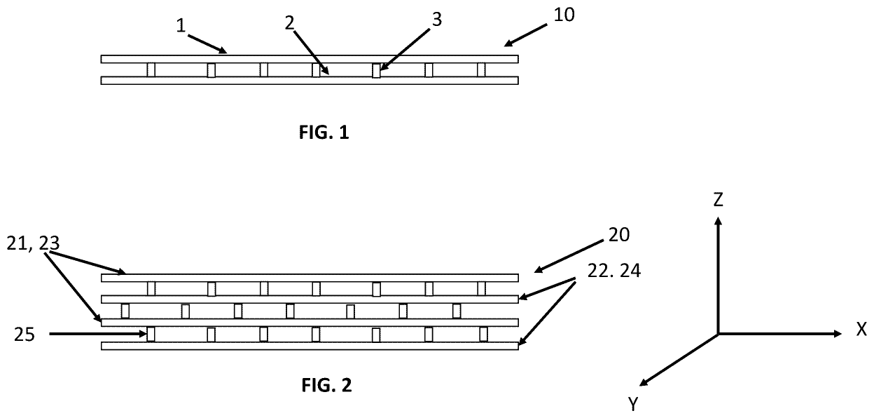

FIG. 1 shows a cross-section of an absorbent nonwoven or composite, generally designated by reference number 10, according to an exemplary embodiment of the present invention. As shown in FIG. 1, the absorbent nonwoven or composite 10 includes at least two layers 1, 2 of nonwoven, tissue, composite of nonwoven and/or tissue, or combinations thereof. In this example, the layers 1, 2 are 50 gsm fibrous nonwoven. A bottom side of layer 1 is attached to a top side of layer 2 by attachment lines of hot melt adhesive 3 that are 2 mm wide as measured in the cross-direction (X-direction), spaced 20 mm apart along the cross-direction. The length of the absorbent nonwoven or composite as measured in the machine direction (Y-direction) is 30 cm and the width as measured in the cross-direction is 10 cm. The thickness of the absorbent nonwoven or composite as measured in the Z-direction is 2 mm. The absorbent nonwoven or composite contains 50% SAP by weight of the absorbent nonwoven or composite.

FIG. 2 shows a cross-section of another absorbent nonwoven, tissue or composite, generally designated by reference number 20, according to an exemplary embodiment of the present invention. As shown in FIG. 2, layers 21 and 23 are 50 gsm fibrous nonwovens and layers 22 and 24 are 20 gsm tissue. A bottom side of layers 21 and 23 are attached to a top side of layers 22 and 24 by attachment lines of hot melt adhesive 25 2 mm wide as measured in the cross-direction, spaced 20 mm apart along the cross-direction. The length of the absorbent nonwoven or composite as measured in the machine-direction is 30 cm and the width as measured in the cross-direction is 10 cm. The thickness of the absorbent nonwoven or composite as measured in the Z-direction is 2 mm. The absorbent nonwoven or composite contains 60% SAP by weight of the absorbent nonwoven or composite.

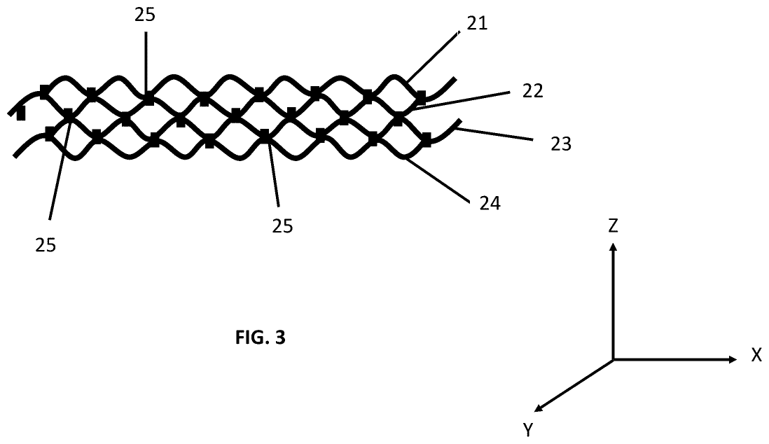

FIG. 3 shows a cross-section of the sample of FIG. 2 when wet, displaying the honeycomb appearance that further enables SAP particles to swell.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

We claim:

1. A nonwoven or composite comprising:
at least two layers with each layer being made of at least one of nonwoven or tissue and having a lower side and an upper side, wherein the lower side of a first layer of the at least two layers is attached to the upper side of an adjacent second layer of the at least two layers with a pattern of continuous attachment lines extending in a machine direction across a cross direction width of the nonwoven or composite.

2. The nonwoven or composite according to claim 1 wherein the attachment lines are from 1 to 3 mm wide and are spaced from 10 to 30 mm apart along the cross direction.

3. The nonwoven or composite according to claim 2 further comprising from 10 to 80% superabsorbent polymer particles by weight of the nonwoven or composite.

4. The nonwoven or composite according to claim 3 wherein a basis weight of the nonwoven or composite ranges from 15 g/m² to 400 g/m².

5. The nonwoven or composite according to claim 4 wherein a dry thickness of the nonwoven or composite ranges from 1 mm to 5 mm and the thickness of the nonwoven or composite when wet ranges from 10 mm to 125 mm.

6. An absorbent product comprising:
a nonwoven or composite comprising:
at least two layers with each layer being made of at least one of nonwoven or tissue and having a lower side and an upper side, wherein the lower side of a first layer of the at least two layers is attached to the upper side of an adjacent second layer of the at least two layers with a pattern of continuous attachment lines extending in a machine direction across a cross direction width of the nonwoven or composite;
a backsheet; and
a topsheet.

7. The absorbent product according to claim 6 wherein the attachment lines are from 1 to 3 mm wide and are spaced from 10 to 30 mm apart along the cross direction.

8. The absorbent product according to claim 7 further comprising from 10 to 80% superabsorbent polymer particles by weight of the nonwoven or composite.

9. The absorbent product according to claim 8 wherein a basis weight of the nonwoven or composite ranges from 50 g/m² to 400 g/m².

10. The absorbent product according to claim 9 wherein a dry thickness of the nonwoven or composite ranges from 1 mm to 5 mm and the thickness of the nonwoven or composite when wet ranges from 10 mm to 125 mm.
